(19) United States
(12) Patent Application Publication
(10) Pub. No.: US 2009/0148871 A1
(43) Pub. Date: Jun. 11, 2009

(54) Interdependent Assays for Detecting Two or More Analytes of Interest in A Test Sample

(76) Inventors: Phillip G. Mattingly, Third Lake, IL (US); Maciej B. Adamczyk, Gurnee, IL (US); Roy Jeffrey Brashear, Mundelein, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/953,261

(22) Filed: Dec. 10, 2007

(51) Int. Cl. C12Q 1/60; C12Q 1/26; C12Q 1/28

(52) U.S. Cl. 435/11; 435/25; 435/28

(57) ABSTRACT

The present invention relates to interdependent assays and kits for detecting and at least two analytes of interest in a single test sample.

### RELATED APPLICATION INFORMATION

None.

### FIELD OF THE INVENTION

The present invention relates to interdependent assays and kits for detecting or quantifying two or more analytes of interest in a test sample.

### BACKGROUND OF THE INVENTION

Test samples may contain analytes that are biomarkers for existing diseases, syndromes, or physiological abnormalities or that indicate a risk of developing such conditions. Various methods for detecting analytes of interest in test samples (such as serum, plasma, whole blood, etc.) have been developed and put into use to enable the early diagnosis of such conditions and for confirming the effects of therapy. For the purpose of qualitative or quantitative detection of an analyte in a test sample, certain detectable compounds (also known as detectable labels or signal generating compounds) are used. Typically, these detectable compounds are capable of being used to generate detectable signals in the presence of one or more analytes in a test sample. In certain instances, these detectable compounds are attached to substances that have a certain affinity for the analyte to be detected and quantified. For example, an antibody can be conjugated to a detectable compound (the labeled antibody is referred to herein as a “conjugate”). The conjugate can then be used to detect and quantify the amount of an antigen of interest in a test sample. In other instances, however, the detectable compound is simply added to the test sample alone, not attached or conjugated to another substance (such as an antibody). Regardless of whether a detectable compound is attached or conjugated to another substance or used alone, once added to the test sample, the compound is activated and the signal detected. As a result, a determination of the presence of an analyte and the amount of the analyte contained in a test sample can be readily determined.

Biomarkers may be endogenous substances such as enzymes, proteins, peptides, glycoproteins, hormones, lipids, nucleic acids or sugars. Alternatively, biomarkers may be exogenous substances such as infectious agents, the byproducts of infectious agents, drugs and drug metabolites, or environmental toxins. In certain instances, two or more biomarkers are used to define a clinical state. For example, the analytes glucose, hemoglobin AIC, insulin and anti-insulin IgG are used in defining the clinical state of a patient suffering from diabetes. Other examples include the group of analytes comprising TSH, T-4, T-uptake, T-3, TG, TPO, anti-TG and anti-TPO IgG used in defining the clinical state of a patient suffering from a thyroid disorder. The group comprising apolipoprotein A1, apolipoprotein B, BNP, CK-MB, CRP, cholesterol, choline, HDL, homocysteine, LDL, myoglobin, myeloperoxidase, triglycerides, and troponin are used in defining the clinical state of a patient suffering from a cardiovascular pathology.

In such instances, the biomarkers are clinically related but are typically assessed by independent analytical procedures. It would be more convenient and cost effective if clinically related analytes were synergistically analyzed using interdependent analytical procedures. An example of one such clinically related pair of analytes is the haloperoxidase, myeloperoxidase (MPO) and choline.

Haloperoxidases are a group of enzymes that are able to catalyse the halogenation of organic compounds. Specifically, haloperoxidases oxidize halides, namely, chloride (Cl⁻), bromide (Br⁻), or (I⁻) but not fluoride (Fl⁻), in the presence of a peroxide, such as hydrogen peroxide (H₂O₂), to hypohalous acid as shown below:

H₂O₂+X⁻+H⁺→H₂O+HOX (where X is the halide Cl⁻, Br⁻, or I⁻).

If a nucleophilic acceptor is present, a reaction will occur with HOX whereby a diversity of halogenated reaction products may be formed.

Haloperoxidases have been isolated from various organisms, such as, mammals, marine animals, plants, algae, lichen, fungi and bacteria. In addition to the halogenation of organic compounds, haloperoxidases have been shown to carry out sulfoxidation, epoxidation, oxidation of indoles and other specific reactions with a range of compounds. Haloperoxidases are named according to the oxidation of the most electrophilic halide that they are able to catalyze. For example, bromoperoxidases are able to oxidize iodide and bromide. Chloroperoxidases are able to oxidize chloride.

Three different groups of haloperoxidases are known. These groups are heme-thiolate containing haloperoxidases (such as chloroperoxidases from Caldariomyces fumao, canine myeloperoxidase, and a peroxidase isolated from Notomastus lobatus, myelo- and eosinophil peroxidases from human white blood cells, bovine lacto- and human thyroid peroxidases (See, Jennifer Littlechild, Current Opinion in Chemical Biology, 3:28-34 (1999) and Hofrichter, M., et al., Appl. Microbiol. Biotechnol., 71:276-288 (2006)), vanadium-containing haloperoxidases (such as vanadium bromoperoxidases from Xantheria parietina and Ascophyllum nodosum and vanadium chloroperoxidases from Caldariomyces inaequalis and Drechslera biseptate) (See, Simons, B., et al., Eur. J. Biochem., 299:566-574 (1995)), and metal-free haloperoxidases (such as, chloroperoxidases A2 from Streptomyces aureofaciens, Streptomyces lividans and Pseudomonas fluorescens (See, Jennifer Littlechild, Current Opinion in Chemical Biology, 3:28-34 (1999)).

It is known that certain types of cells generate hydrogen peroxide. Moreover, many of the same cells or types of cells are also known to secrete haloperoxidases. For example, white blood cells are known to generate hydrogen peroxide and to secrete myeloperoxidase. In the presence of hydrogen peroxide, myeloperoxidase catalyzes the oxidation of chloride to hypochlorous acid (HOCl). HOCl is a potent cytotoxin for bacteria, viruses and fungi. The generation of HOCl by white blood cells plays a key role in host defenses against invading pathogens. However, oxidant production by phagocytic white cells is also potentially deleterious and is believed to represent an important pathway for tissue damage in disorders ranging from arthritis to ischemia reperfusion injury to cancer.

Oxidative injury is believed to be of central importance in promoting atherosclerotic heart disease. One risk factor in atherosclerosis is elevated levels of low density lipoprotein (“LDL”). In vitro, LDL fails to exert effects that would promote heart disease in vivo. However, oxidation of LDL, renders the lipoprotein atherogenic. Many lines of evidence indicate that the oxidation of LDL is of central importance in the promotion of heart disease. Oxidized LDL has been isolated from atherosclerotic lesions and antioxidants have been found to retard atherosclerosis in animals.

Elevated levels of haloperoxidases, such as myeloperoxidase (MPO), in subjects with cardiovascular disease, have been associated with arterial inflammation. A number of studies have linked arterial inflammation with an increased risk of cardiovascular events. Additionally, recent studies have shown that serum myeloperoxidase levels are associated with the future risk of coronary artery disease in apparently healthy individuals (See, Marijn C. Meuwese et al., Journal of the American College of Cardiology, 50(2):159-165 (2007)). The measurement in test samples such as blood of the levels of haloperoxidases such as myeloperoxidase are used to predict whether or not an individual is at risk of developing cardiovascular disease, such as coronary heart disease.

Methods for detecting haloperoxidases are described in U.S. patent application Ser. No. 11/842,897, entitled, “Measurement of Haloperoxidase Activity with Chemiluminescent Detection,” the contents of which are herein incorporated by reference. Briefly, the method described in this application employs in part, a known quantity of hydrogen peroxide in conjunction with a chemiluminescent detection reagent to generate a light signal inversely proportional to the concentration of the haloperoxidase in the test sample. The haloperoxidase concentration is determined from a two-dimensional dose response curve.

Choline, a major constituent of cell membrane phospholipids, is important in the study of myocardial ischemia/reperfusion injury and as well as acute coronary syndrome (See, Apple F S, Wu A H, Mair J, Ravkilde J, Panteghini M, Tate J, et al., Clin Chem., 51, 810-24 (2005)). During reperfusion after global ischemia, choline is released in a biphasic manner (See, Bruhl A, Hafner G, Loffelholz K., Life Sci., 75:1609-20 (2004)). Ischemic preconditioning blocks the second phase of choline efflux attributed to the degradation of phospholipids mediated by cytostolic phospholipase A2. Danne, et al. (See, Danne O, Mockel M, Lueders C, Mugge C, Zschunke G A, Lufft H, et al., Am J Cardiol., 91:1060-7 (2003)) have performed a prospective study on cardiac troponin negative patients with suspected acute coronary syndrome relying on the analysis of choline in whole blood by LC-MS. The authors concluded that increased whole blood choline concentrations were predictive of cardiac death and non-fatal cardiac arrest.

Methods for detecting choline in plasma and whole blood include Adamczyk, M., Brashear, R. J., Mattingly, P. G., and Tsatsos, P. H., Anal. Chim. Acta 579, 61-67 (2006)); Adamczyk, M., Brashear, R. J., and Mattingly, P. G., Clin Chem., 52, 2123-2124 (2006); Adamczyk, M., Brashear, R. J., and Mattingly, P. G., Bioorg. Med. Chem. Lett., 16, 2407-2410 (2006)); and in U.S. patent application Ser. No. 11/697,835, entitled, “Acridinium Phenyl Esters Useful in the Analysis of Biological Samples”, the contents of each of the above are herein incorporated by reference in their entirety.

Typically, assays for detecting analytes of interest, such as choline and haloperoxidases, are performed independently. It would be more convenient and cost effective if such assays could be combined into a single interdependent test format. Thereupon, there is a need in the art for methods of detecting or determining the amount of two or more analytes of interest in a single test sample in an interdependent manner.

### SUMMARY OF THE PRESENT INVENTION

In one embodiment, the present invention relates to an interdependent method for detecting at least at least two analytes of interest in a test sample. The method comprises the steps of:

a) contacting a test sample containing a first analyte of interest and a second analyte of interest with at least one analyte-specific enzyme, wherein the first analyte of interest is selected from the group consisting of: galactose, glucose, cholesterol, LDL, HDL, choline, lactic acid, uric acid, phosphatidylcholine, acetylcholine, phosphocholine, CDP-choline, lysophosphatidylcholine, triglycerides and sphingomyelin;

b) adding an acridinium-9-carboxamide to the test sample;

c) adding a basic solution to the test sample to generate a light signal;

d) measuring the light generated from the light signal and calculating the amount of first analyte of interest present in the test sample; and

e) performing a three dimensional dose response surface analysis to calculate the amount of the second analyte of interest in the test sample.

The test sample used in the above method can be whole blood, serum or plasma.

In the above method, the analyte-specific enzyme is a dismutase, dehydrogenase, oxidase, reductase or synthase or a combination of at least one dismutase, dehydrogenase, oxidase, reductase or synthase. Specifically, the analyte-specific enzyme is selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

Additionally, in the above method, the second analyte of interest is a haloperoxidase. Specifically, the haloperoxidase is selected from the group consisting of: myeloperoxidase, thyroperoxidase, eosinoperoxidase, eosinophil peroxidase and lactoperoxidase.

The above method can further comprise the step of quantifying the amount of the first analyte of interest in the test sample by relating the amount of light generated in the test sample by comparison to a standard curve for said analyte. Specifically, the standard curve is generated from solutions of an analyte of a known concentration.

The above method can further comprise the step of quantifying the activity of amount of the second analyte of interest by using a combination of known concentrations of the first analyte of interest and the second analyte of interest.

In the above method, the acridinium-9-carboxamide has a structure according to formula I:

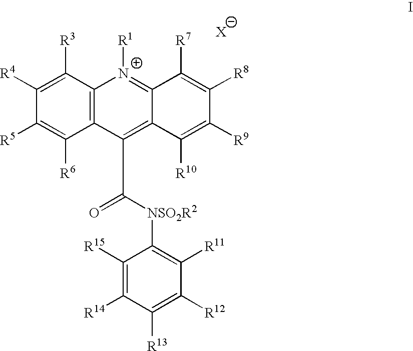

wherein R¹ and R² are each independently selected from the group consisting of: alkyl, alkenyl, alkynyl, aryl or aralkyl, sulfoalkyl and carboxyalkyl, and
wherein R³ through R¹⁵ are each independently selected from the group consisting of: hydrogen; alkyl, alkenyl, alkynyl, aryl or aralkyl, amino, amido, acyl, alkoxyl,
   hydroxyl, carboxyl, halide, nitro, cyano, sulfo, sulfoalkyl, and carboxyalkyl; and

optionally, if present, X^{⊖} is an anion.

In a second embodiment, the present invention relates to an interdependent method for detecting at least at least two analytes of interest in a test sample. The method comprises the steps of:

a) contacting a test sample containing a first analyte of interest and a second analyte of interest with at least one analyte-specific enzyme;

b) sampling the test mixture to obtain a first aliquot containing a portion of the first analyte of interest and the second analyte of interest from the test sample;

c) adding a first acridinium-9-carboxamide to the first aliquot;

d) adding a first basic solution to the first aliquot to generate a light signal;

e) measuring the light generated from the light signal;

f) sampling the test mixture to obtain a second aliquot containing a portion of the first analyte of interest and the second analyte of interest from the test sample;

g) adding a second acridinium-9-carboxamide to the second aliquot;

h) adding a second basic solution to the second aliquot to generate a light signal;

i) measuring the light generated from the light signal in step h); and

j) performing a three dimensional dose response surface analysis using the amount of light measured in steps e) and i)) to calculate the amount of the first and second analytes of interest in the test sample.

The test sample used in the above method can be whole blood, serum or plasma.

In the above method, the first analyte of interest is selected from the group consisting of: galactose, glucose, cholesterol, LDL, HDL, choline, lactic acid, uric acid, phosphatidylcholine, acetylcholine, phosphocholine, CDP-choline, lysophosphatidylcholine, triglycerides and sphingomyelin.

In the above method, the analyte-specific enzyme is a dismutase, dehydrogenase, oxidase, reductase or synthase or a combination of at least one dismutase, dehydrogenase, oxidase, reductase or synthase. Specifically, the analyte-specific enzyme is selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

In the above method, the second analyte of interest is a haloperoxidase. The haloperoxidase is selected from the group consisting of: myeloperoxidase, thyroperoxidase, eosinoperoxidase, eosinophil peroxidase and lactoperoxidase.

In the above method, the first acridinium-9-carboxamide and second acridinium-9-carboxamide each have a structure according to formula I:

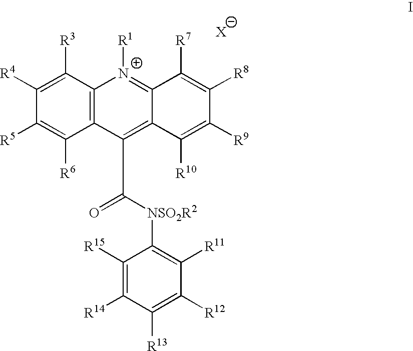

wherein R¹ and R² are each independently selected from the group consisting of: alkyl, alkenyl, alkynyl, aryl or aralkyl, sulfoalkyl and carboxyalkyl, and
wherein R³ through R¹⁵ are each independently selected from the group consisting of: hydrogen; alkyl, alkenyl, alkynyl, aryl or aralkyl, amino, amido, acyl, alkoxyl,
   hydroxyl, carboxyl, halide, nitro, cyano, sulfo, sulfoalkyl, and carboxyalkyl; and

optionally, if present, X^{⊖} is an anion.

In yet another embodiment, the present invention relates to a kit for use in detecting at least two analytes of interest in a test sample. The kit comprises:

a. at least one acridinium-9-carboxamide;

b. at least one basic solution;

c. at least one analyte-specific enzyme or hydrogen peroxide generating enzyme;

d. instructions for detecting the amount of at least one analyte of interest in a test sample; and

e. instructions for performing a dimensional dose response surface analysis to calculate the amount of at least one analyte of interest in the test sample.

In the above kit, the at least one analyte-specific enzyme or hydrogen peroxide generating enzyme is a dismutase, dehydrogenase, oxidase, reductase or synthase or a combination of at least one dismutase, dehydrogenase, oxidase, reductase or synthase. Specifically, the at least one analyte-specific enzyme or hydrogen peroxide generating enzyme is selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

In the above kit, the acridinium-9-carboxamide has a structure according to formula I:

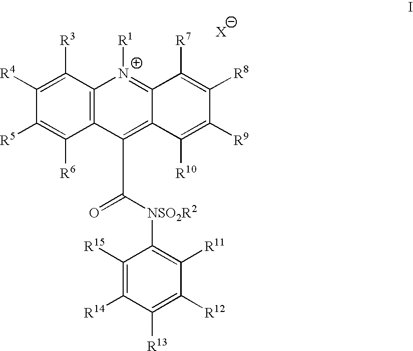

wherein R¹ and R² are each independently selected from the group consisting of: alkyl, alkenyl, alkynyl, aryl or aralkyl, sulfoalkyl and carboxyalkyl, and
wherein R³ through R¹⁵ are each independently selected from the group consisting of: hydrogen; alkyl, alkenyl, alkynyl, aryl or aralkyl, amino, amido, acyl, alkoxyl,
   hydroxyl, carboxyl, halide, nitro, cyano, sulfo, sulfoalkyl, and carboxyalkyl; and

optionally, if present, X^{⊖} is an anion.

In the above kit, at least one analyte is selected from the group consisting of: galactose, glucose, cholesterol, LDL, HDL, choline, lactic acid, uric acid, phosphatidylcholine, acetylcholine, phosphocholine, CDP-choline, lysophosphatidylcholine, triglycerides and sphingomyelin.

Alternatively, the at least one analyte is a haloperoxidase. Specifically, the haloperoxidase is selected from the group consisting of: myeloperoxidase, thyroperoxidase, eosinoperoxidase, eosinophil peroxidase and lactoperoxidase.

In yet another embodiment, the present invention relates to a kit for use in detecting at least two analytes of interest in a test sample. The kit comprises:

a. at least two acridinium-9-carboxamides;

b. at least two basic solutions;

c. at least one analyte-specific enzyme or hydrogen generating enzyme; and

d. instructions for performing a dimensional dose response surface analysis to calculate the amount of at least two analytes of interest in the test sample.

In the above kit, the at least one analyte-specific enzyme or hydrogen generating enzyme is a dismutase, dehydrogenase, oxidase, reductase or synthase or a combination of at least one dismutase, dehydrogenase, oxidase, reductase or synthase. Specifically, the at least one analyte-specific enzyme or hydrogen peroxide generating enzyme is selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

In the above kit, each of the acridinium-9-carboxamides has a structure according to formula I:

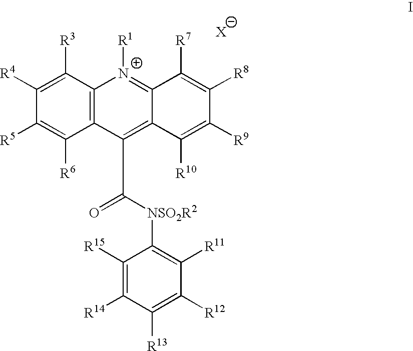

wherein R¹ and R² are each independently selected from the group consisting of: alkyl, alkenyl, alkynyl, aryl or aralkyl, sulfoalkyl and carboxyalkyl, and
wherein R³ through R¹⁵ are each independently selected from the group consisting of: hydrogen; alkyl, alkenyl, alkynyl, aryl or aralkyl, amino, amido, acyl, alkoxyl,
   hydroxyl, carboxyl, halide, nitro, cyano, sulfo, sulfoalkyl, and carboxyalkyl; and

optionally, if present, X^{⊖} is an anion.

In the above kit, at least one analyte is selected from the group consisting of: galactose, glucose, cholesterol, LDL, HDL, choline, lactic acid, uric acid, phosphatidylcholine, acetylcholine, phosphocholine, CDP-choline, lysophosphatidylcholine, triglycerides and sphingomyelin.

Alternatively, the at least one analyte is a haloperoxidase. Specifically, the haloperoxidase is selected from the group consisting of: myeloperoxidase, thyroperoxidase, eosinoperoxidase, eosinophil peroxidase and lactoperoxidase.

In the above kit, the at least two acridinium-9-carboxamides are each different from one another. Alternatively, the at least two acridinium-9-carboxamides are the same.

In the above kit, the at least two basic solutions are different from each other. Alternatively, the at least two basic solutions are the same.

### BRIEF DESCRIPTION OF THE FIGURES

FIG. 1 shows a three dimensional (“3-D”) dose-response surface for analysis of choline and myeloperoxidase determined pursuant to Example 1.

### DETAILED DESCRIPTION OF THE PRESENT INVENTION

The present invention provides interdependent assays and kits for detecting and quantifying at least two analytes of interest in a test sample.

A. Definitions

As used herein, the term “acyl” refers to a —C(O)Rₐ group where Rₐ is hydrogen, alkyl, cycloalkyl, cycloalkylalkyl, phenyl or phenylalkyl. Representative examples of acyl include, but are not limited to, formyl, acetyl, cylcohexylcarbonyl, cyclohexylmethylcarbonyl, benzoyl, benzylcarbonyl and the like.

As used herein, the term “alkenyl” means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the term “alkyl” means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

As used herein, the term “alkyl radical” means any of a series of univalent groups of the general formula CₙH₂ₙ₊₁ derived from straight or branched chain hydrocarbons.

As used herein, the term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

As used herein, the term “alkynyl” means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

As used herein, the term “amido” refers to an amino group attached to the parent molecular moiety through a carbonyl group (wherein the term “carbonyl group” refers to a —C(O)— group).

As used herein, the term “amino” means —NR_{b}R_{c}, wherein R_{b} and R_{c} are independently selected from the group consisting of hydrogen, alkyl and alkylcarbonyl.

As used herein, the phrases “analyte-specific enzyme” and “hydrogen peroxide generating enzyme”, which are used interchangeable herein, refer to an enzyme which produces a peroxide, including, dismutases, dehydrogenases, oxidases, reductases, synthases or combinations thereof. Exemplary analyte-specific enzymes/hydrogen peroxide generating enzymes which produce a peroxide are listed below in Table A. Many analyte-specific enzymes/hydrogen peroxide generating enzymes that produce a peroxide are known in the art. For example, analyte-specific enzymes/hydrogen peroxide generating enzymes which produces a peroxide can be conveniently found in on the on the World Wide Web at the Enzyme Nomenclature Database and the Enzyme Database (developed at Trinity College in Dublin, Ireland).

TABLE A

| | IUBMB ENZYME | PREFERRED |
|---|---|---|
| ACCEPTED COMMON NAME | NOMENCLATURE | SUBSTRATE |
| (R)-6-hydroxynicotine oxidase | EC 1.5.3.6 | (R)-6-hydroxynicotine |
| (S)-2-hydroxy acid oxidase | EC 1.1.3.15 | S)-2-hydroxy acid |
| (S)-6-hydroxynicotine oxidase | EC 1.5.3.5 | (S)-6-hydroxynicotine |
| 3-aci-nitropropanoate oxidase | EC 1.7.3.5 | 3-aci-nitropropanoate |
| 3-hydroxyanthranilate oxidase | EC 1.10.3.5 | 3-hydroxyanthranilate |
| 4-hydroxymandelate oxidase | EC 1.1.3.19 | (S)-2-hydroxy-2-(4- |
| | | hydroxyphenyl)acetate |
| 6-hydroxynicotinate dehydrogenase | EC 1.17.3.3 | 6-hydroxynicotinate |
| Abscisic-aldehyde oxidase | EC 1.2.3.14 | abscisic aldehyde |
| acyl-CoA oxidase | EC 1.3.3.6 | acyl-CoA |
| Alcohol oxidase | EC 1.1.3.13 | a primary alcohol |
| aldehyde oxidase | EC 1.2.3.1 | an aldehyde |
| amine oxidase | | |
| amine oxidase (copper-containing) | EC 1.4.3.6 | primary monoamines, |
| | | diamines and histamine |
| amine oxidase (flavin-containing) | EC 1.4.3.4 | a primary amine |
| aryl-alcohol oxidase | EC 1.1.3.7 | an aromatic primary alcohol |
| | | (2-naphthyl)methanol |
| | | 3-methoxybenzyl alcohol |
| aryl-aldehyde oxidase | EC 1.2.3.9 | an aromatic aldehyde |
| catechol oxidase | EC 1.1.3.14 | catechol |
| cholesterol oxidase | EC 1.1.3.6 | cholesterol |
| Choline oxidase | EC 1.1.3.17 | choline |
| columbamine oxidase | EC 1.21.3.2 | columbamine |
| cyclohexylamine oxidase | EC 1.4.3.12 | cyclohexylamine |
| cytochrome c oxidase | EC 1.9.3.1 | |
| D-amino-acid oxidase | EC 1.4.3.3 | a D-amino acid |
| D-arabinono-1,4-lactone oxidase | EC 1.1.3.37 | D-arabinono-1,4-lactone |
| D-arabinono-1,4-lactone oxidase | EC 1.1.3.37 | D-arabinono-1,4-lactone |
| D-aspartate oxidase | EC 1.4.3.1 | D-aspartate |
| D-glutamate oxidase | EC 1.4.3.7 | D-glutamate |
| D-glutamate(D-aspartate) oxidase | EC 1.4.3.15 | D-glutamate |
| dihydrobenzophenanthridine oxidase | EC 1.5.3.12 | dihydrosanguinarine |
| dihydroorotate oxidase | EC 1.3.3.1 | (S)-dihydroorotate |
| dihydrouracil oxidase | EC 1.3.3.7 | 5,6-dihydrouracil |
| dimethylglycine oxidase | EC 1.5.3.10 | N,N-dimethylglycine |
| D-mannitol oxidase | EC 1.1.3.40 | mannitol |
| ecdysone oxidase | EC 1.1.3.16 | ecdysone |
| ethanolamine oxidase | EC 1.4.3.8 | ethanolamine |
| galactose oxidase | EC 1.1.3.9 | D-galactose |
| Glucose oxidase | EC 1.1.3.4 | β-D-glucose |
| glutathione oxidase | EC 1.8.3.3 | glutathione |
| glycerol-3-phosphate oxidase | EC 1.1.3.21 | sn-glycerol 3-phosphate |
| Glycine oxidase | EC 1.4.3.19 | glycine |
| glyoxylate oxidase | EC 1.2.3.5 | glyoxylate |
| hexose oxidase | EC 1.1.3.5 | D-glucose, |
| | | D-galactose |
| | | D-mannose |
| | | maltose |
| | | lactose |
| | | cellobiose |
| Hydroxyphytanate oxidase | EC 1.1.3.27 | L-2-hydroxyphytanate |
| indole-3-acetaldehyde oxidase | EC 1.2.3.7 | (indol-3-yl)acetaldehyde |
| lactic acid oxidase | | Lactic acid |
| L-amino-acid oxidase | EC 1.4.3.2 | an L-amino acid |
| L-aspartate oxidase | EC 1.4.3.16 | L-aspartate |
| L-galactonolactone oxidase | EC 1.3.3.12 | L-galactono-1,4-lactone |
| L-glutamate oxidase | EC 1.4.3.11 | L-glutamate |
| L-gulonolactone oxidase | EC 1.1.3.8 | L-gulono-1,4-lactone |
| L-lysine 6-oxidase | EC 1.4.3.20 | L-lysine |
| L-lysine oxidase | EC 1.4.3.14 | L-lysine |
| long-chain-alcohol oxidase | EC 1.1.3.20 | A long-chain-alcohol |
| L-pipecolate oxidase | EC 1.5.3.7 | L-pipecolate |
| L-sorbose oxidase | EC 1.1.3.11 | L-sorbose |
| malate oxidase | EC 1.1.3.3 | (S)-malate |
| methanethiol oxidase | EC 1.8.3.4 | methanethiol |
| monoamino acid oxidase | | |
| N⁶-methyl-lysine oxidase | EC 1.5.3.4 | 6-N-methyl-L-lysine |
| N-acylhexosamine oxidase | EC 1.1.3.29 | N-acetyl-D-glucosamine |
| | | N-glycolylglucosamine |
| | | N-acetylgalactosamine |
| | | N-acetylmannosamine. |
| NAD(P)H oxidase | EC 1.6.3.1 | NAD(P)H |
| nitroalkane oxidase | EC 1.7.3.1 | a nitroalkane |
| N-methyl-L-amino-acid oxidase | EC 1.5.3.2 | an N-methyl-L-amino acid |
| nucleoside oxidase | EC 1.1.3.39 | adenosine |
| Oxalate oxidase | EC 1.2.3.4 | oxalate |
| polyamine oxidase | EC 1.5.3.11 | 1-N-acetylspermine |
| polyphenol oxidase | EC 1.14.18.1 | |
| polyvinyl-alcohol oxidase | EC 1.1.3.30 | polyvinyl alcohol |
| prenylcysteine oxidase | EC 1.8.3.5 | an S-prenyl-L-cysteine |
| Protein-lysine 6-oxidase | EC 1.4.3.13 | peptidyl-L-lysyl-peptide |
| putrescine oxidase | EC 1.4.3.10 | butane-1,4-diamine |
| pyranose oxidase | EC 1.1.3.10 | D-glucose |
| | | D-xylose |
| | | L-sorbose |
| | | D-glucono-1,5-lactone |
| pyridoxal 5′-phosphate synthase | EC 1.4.3.5 | pyridoxamine 5′- |
| | | phosphate |
| pyridoxine 4-oxidase | EC 1.1.3.12 | pyridoxine |
| pyrroloquinoline-quinone synthase | EC 1.3.3.11 | 6-(2-amino-2- |
| | | carboxyethyl)-7,8-dioxo- |
| | | 1,2,3,4,5,6,7,8- |
| | | octahydroquinoline-2,4- |
| | | dicarboxylate |
| pyruvate oxidase | EC 1.2.3.3 | pyruvate |
| pyruvate oxidase (CoA-acetylating) | EC 1.2.3.6 | pyruvate |
| reticuline oxidase | EC 1.21.3.3 | reticuline |
| retinal oxidase | EC 1.2.3.11 | retinal |
| rifamycin-B oxidase | EC 1.10.3.6 | rifamycin-B |
| sarcosine oxidase | EC 1.5.3.1 | sarcosine |
| secondary-alcohol oxidase | EC 1.1.3.18 | a secondary alcohol |
| sulfite oxidase | EC 1.8.3.1 | sulfite |
| superoxide dismutase | EC 1.15.1.1 | superoxide |
| superoxide reductase | EC 1.15.1.2 | superoxide |
| tetrahydroberberine oxidase | EC 1.3.3.8 | (S)-tetrahydroberberine |
| thiamine oxidase | EC 1.1.3.23 | thiamine |
| tryptophan α,β-oxidase | EC 1.3.3.10 | L-tryptophan |
| urate oxidase (uricase, uric acid | EC 1.7.3.3 | uric acid |
| oxidase) | | |
| Vanillyl-alcohol oxidase | EC 1.1.3.38 | vanillyl alcohol |
| xanthine oxidase | EC 1.17.3.2 | xanthine |
| xylitol oxidase | EC 1.1.3.41 | xylitol |

As used herein, the term “anion” refers to an anion of an inorganic or organic acid, such as, but not limited to, hydrochloric acid, hydrobromic acid, sulfuric acid, methane sulfonic acid, formic acid, acetic acid, oxalic acid, succinic acid, tartaric acid, mandelic acid, fumaric acid, lactic acid, citric acid, glutamic acid, aspartic acid, phosphate, trifluoromethansulfonic acid, trifluoroacetic acid and fluorosulfonic acid and any combinations thereof.

As used herein, the term “aralkyl” means an aryl group appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

As used herein, the term “aryl” means a phenyl group, or a bicyclic or tricyclic fused ring system wherein one or more of the fused rings is a phenyl group. Bicyclic fused ring systems are exemplified by a phenyl group fused to a cycloalkenyl group, a cycloalkyl group, or another phenyl group. Tricyclic fused ring systems are exemplified by a bicyclic fused ring system fused to a cycloalkenyl group, a cycloalkyl group, as defined herein or another phenyl group. Representative examples of aryl include, but are not limited to, anthracenyl, azulenyl, fluorenyl, indanyl, indenyl, naphthyl, phenyl, and tetrahydronaphthyl. The aryl groups of the present invention can be optionally substituted with one-, two, three, four, or five substituents independently selected from the group consisting of alkoxy, alkyl, carboxyl, halo, and hydroxyl.

As used herein, the term “carboxy” or “carboxyl” refers to —CO₂H or —CO₂⁻.

As used herein, the term “carboxyalkyl” refers to a —(CH₂)ₙCO₂H or —(CH₂)ₙCO₂⁻ group where n is from 1 to 10.

As used herein, the term “cyano” means a —CN group.

As used herein, the term “cycloalkenyl” refers to a non-aromatic cyclic or bicyclic ring system having from three to ten carbon atoms and one to three rings, wherein each five-membered ring has one double bond, each six-membered ring has one or two double bonds, each seven- and eight-membered ring has one to three double bonds, and each nine- to ten-membered ring has one to four double bonds. Representative examples of cycloalkenyl groups include cyclohexenyl, octahydronaphthalenyl, norbornylenyl, and the like. The cycloalkenyl groups can be optionally substituted with one, two, three, four, or five substituents independently selected from the group consisting of alkoxy, alkyl, carboxyl, halo, and hydroxyl.

As used herein, the term “cycloalkyl” refers to a saturated monocyclic, bicyclic, or tricyclic hydrocarbon ring system having three to twelve carbon atoms. Representative examples of cycloalkyl groups include cyclopropyl, cyclopentyl, bicyclo[3.1.1]heptyl, adamantyl, and the like. The cycloalkyl groups of the present invention can be optionally substituted with one, two, three, four, or five substituents independently selected from the group consisting of alkoxy, alkyl, carboxyl, halo, and hydroxyl.

As used herein, the term “cycloalkylalkyl” means a —R_{d}Rₑ group where R_{d} is an alkylene group and Rₑ is cycloalkyl group. A representative example of a cycloalkylalkyl group is cyclohexylmethyl and the like.

As used herein, the term “halogen” means a —Cl, —Br, —I or —F; the term “halide” means a binary compound, of which one part is a halogen atom and the other part is an element or radical that is less electronegative than the halogen, e.g., an alkyl radical.

As used herein, the term “hydroxyl” means an —OH group.

As used herein, the term “nitro” means a —NO₂ group.

As used herein, the term “oxoalkyl” refers to —(CH₂)ₙC(O)Rₐ, where Rₐ is hydrogen, alkyl, cycloalkyl, cycloalkylalkyl, phenyl or phenylalkyl and where n is from 1 to 10.

As used herein, the term “phenylalkyl” means an alkyl group which is substituted by a phenyl group.

As used herein, the term “sulfo” means a —SO₃H group.

As used herein, the term “sulfoalkyl” refers to a —(CH₂)ₙSO₃H or —(CH₂)ₙSO₃⁻ group where n is from 1 to 10.

As used herein, the term “test sample” generally refers to a biological material being tested for and/or suspected of containing an analyte of interest, such as galactose, glucose, cholesterol, LDL, HDL, choline, lactic acid, uric acid, phosphatidylcholine, acetylcholine, phosphocholine, CDP-choline, lysophosphatidylcholine, triglycerides, phospholipase A2, phosholipase D, lysophosholipase D and sphingomyelin. Preferably, the test sample also contains one or more haloperoxidases, such as, but not limited to, myeloperoxidase, thyroperoxidase (TPO), eosinoperoxidase (EPO, eosinophil peroxidase), lactoperoxidase or any combinations thereof. Optionally, the test sample contains cells which produce or secrete one or more haloperoxidases, such as, but not limited to, myeloperoxidase, thyroperoxidase (TPO), eosinoperoxidase (EPO, eosinophil peroxidase), lactoperoxidase or any combinations thereof. The test sample may be derived from any biological source, such as, a physiological fluid, including, but not limited to, whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen and so forth. Besides physiological fluids, other liquid samples may be used such as water, food products, and so forth, for the performance of environmental or food production assays. In addition, a solid material suspected of containing the analyte may be used as the test sample. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

As used in this specification and the appended claims, the singular forms “a”, “an”, and “the” include plural references unless the context clearly dictates otherwise. Thus, for example, a reference to “the method” includes one or more methods, and/or steps of the type described herein and/or which will become apparent to those persons skilled in the art upon reading this disclosure and so forth.

B. Interdependent Assay for Detecting or Quantifying at Least Two Analytes in a Test Sample

In general, the present invention relates to an interdependent assay for detecting or quantifying at least two different analytes in a test sample. Preferably, the test sample contains at least a first analyte of interest and a second analyte of interest.

As alluded to above, the assay or method of the present invention involves obtaining a test sample containing at least two analytes of interest from a subject. A subject from which a test sample can be obtained is any vertebrate. Preferably, the vertebrate is a mammal. Examples of mammals include, but are not limited to, dogs, cats, rabbits, mice, rats, goats, sheep, cows, pigs, horses, non-human primates and humans. The test sample can be obtained from the subject using routine techniques known to those skilled in the art. Preferably, one analyte of interest is galactose, glucose, cholesterol, LDL, HDL, choline, lactic acid, uric acid, phosphatidylcholine, acetylcholine, phosphocholine, CDP-choline or sphingomyelin. Preferably, another analyte of interest is one or more haloperoxidases, such as, but not limited to, myeloperoxidase, thyroperoxidase (TPO), eosinoperoxidase (EPO, eosinophil peroxidase), lactoperoxidase or any combinations thereof. Optionally, the test sample can contain cells which produce or secrete one or more haloperoxidases, such as, but not limited to, myeloperoxidase, thyroperoxidase (TPO), eosinoperoxidase (EPO, eosinophil peroxidase), lactoperoxidase or any combinations thereof.

In one embodiment, after the test sample containing at least two analytes of interest is obtained from a subject, the concentration of one of the analytes (which will be referred to as the “first analyte” of interest) is determined. For example, if the first analyte of interest to be detected or quantified is choline, the analyte-specific enzyme is choline oxidase. Alternatively, the first analyte may be glucose and the analyte-specific enzyme glucose oxidase; the first analyte may be cholesterol and the analyte-specific enzyme cholesterol oxidase; the first analyte may be HDL and the analyte-specific enzyme cholesterol oxidase; the first analyte may be triglycerides and the analyte-specific enzyme glycerol-3-phosphate oxidase; the first analyte may be lactic acid and the analyte-specific enzyme lactate oxidase; or the first analyte may be uric acid and the analyte-specific enzyme uric oxidase. Preferably, the amount of the analyte-specific enzyme that can be added to the test sample is from about 0.0001 unit/mL to about 10,000 units/mL.

The second analyte to be determined can be a haloperoxidase. If the second analyte to be determined is a haloperoxidase, then the determination is based on haloperoxidase activity. As used herein, the “haloperoxidase activity” refers to the turnover or consumption of a substrate based on a quantifiable amount (e.g., mass) of a haloperoxidase. In other words, haloperoxidase activity refers to the amount of haloperoxidase needed to convert or change a substrate into the requisite product in a given time. The determination of haloperoxidase activity requires hydrogen peroxide that is provided or is generated upon addition of an analyte-specific enzyme to the test sample containing the first analyte. For example, in one aspect, hydrogen peroxide is generated in situ in the test sample or provided or supplied to the test sample before the addition of the herein described acridinium-9-carboxamide. In a second aspect, the hydrogen peroxide is generated in situ in the test ample or provided or supplied to the test sample simultaneously with the herein-described acridinium-9-carboxamide. In a third aspect, hydrogen peroxide is generated in situ or provided or supplied to the test sample after the above-described acridinium-9-carboxamide is added to the test sample.

For example, if the haloperoxidase activity to be detected or determined is the activity of myeloperoxidase, then the amount of hydrogen peroxide that can be generated in situ or provided or supplied to the test sample is from about 0.0001 micromolar to about 200 micromolar.

The time at which the at least one analyte-specific enzyme or hydrogen peroxide generating enzyme is added to the test sample is not critical, provided that it is added before the addition of the at least one acridinium carboxamide having the structure according to formula I, which will be discussed in more detail below.

Preferably, the at least one analyte-specific enzyme or hydrogen peroxide generating enzyme is at least one oxidase. Oxidases can be used to generate hydrogen peroxide in situ in the test sample. The peroxide that is generated by the addition of the at least hydrogen peroxide generating enzyme can then be converted to an end product having a distinct chemiluminescent emission to indicate the presence of at least one first analyte, such as choline and subsequently, the haloperoxidase second analyte

After the addition of at least one analyte-specific enzyme or hydrogen peroxide generating enzyme to the test sample, at least one acridinium carboxamide is added to the test sample. Preferably, the acridinium carboxamide is an acridinium-9-carboxamide, including optionally an acridinium-9-carboamide having a structure according to formula I shown below:

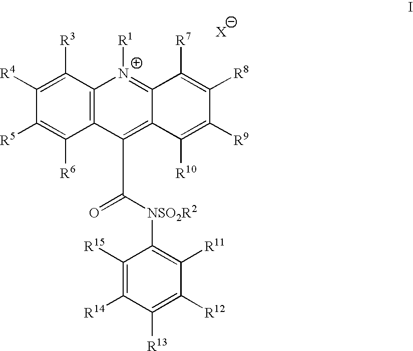

wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl,
   hydroxyl, carboxyl, halogen, halide, nitro, cyano, sulfo, sulfoalkyl, carboxyalkyl and oxoalkyl;
   and further wherein any of the alkyl, alkenyl, alkynyl, aryl or aralkyl may contain one or more heteroatoms; and

optionally, if present, X^{⊖} is an anion.

Methods for preparing acridinium 9-carboxamides are described in Mattingly, P. G. J. Biolumin. Chemilumin., 6, 107-14; (1991); Adamczyk, M.; Chen, Y.-Y., Mattingly, P. G.; Pan, Y. J. Org. Chem., 63, 5636-5639 (1998); Adamczyk, M.; Chen, Y.-Y.; Mattingly, P. G.; Moore, J. A.; Shreder, K. Tetrahedron, 55, 10899-10914 (1999); Adamczyk, M.; Mattingly, P. G.; Moore, J. A.; Pan, Y. Org. Lett., 1, 779-781 (1999); Adamczyk, M.; Chen, Y.-Y.; Fishpaugh, J. R.; Mattingly, P. G.; Pan, Y.; Shreder, K.; Yu, Z. Bioconjugate Chem., 11, 714-724 (2000); Mattingly, P. G.; Adamczyk, M. In Luminescence Biotechnology: Instruments and Applications; Dyke, K. V. Ed.; CRC Press: Boca Raton, pp. 77-105 (2002); Adamczyk, M.; Mattingly, P. G.; Moore, J. A.; Pan, Y. Org. Lett., 5, 3779-3782 (2003); and U.S. Pat. Nos. 5,468,646, 5,543,524, and 5,783,699 (each incorporated herein by reference in their entireties for their teachings regarding same).

The timing and order in which the acridinium-9-carboxamide is supplied to the test sample is not critical provided that it is added after the addition of the at least one analyte-specific enzyme or hydrogen peroxide generating enzyme and prior to the addition of at least one basic solution, which will be discussed in more detail below.

After the addition of the acridinium-9-carboxamide having the structure according to formula I to the test sample, at least one basic solution is added to the test sample in order to generate a detectable signal, namely, a first chemiluminescent signal. The basic solution is a solution that contains at least one base and that has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate and calcium bicarbonate. The amount of basic solution added to the test sample depends on the concentration of the basic solution used in the assay. Based on the concentration of the basic solution used, one skilled in the art could easily determine the amount of basic solution to be used in the method. Chemiluminescent signals generated can be detected using routine techniques known to those skilled in the art.

Thus, the first chemiluminescent signal generated after the addition of a basic solution, indicates the presence of the first analyte of interest, such as, for example, choline. The amount of the first analyte in the test sample can be quantified based on the intensity of the first signal generated. Specifically, the amount of first analyte contained in a test sample is proportional (such as choline) to the first signal generated. Specifically, the amount of the analyte of interest present can be quantified based on comparing the amount of light generated to a standard curve for the analyte or by comparison to a reference standard. The standard curve can be generated using serial dilutions or solutions of analyte of interest of known concentration, by mass spectroscopy, gravimetrically and by other techniques known in the art.

After the first analyte of interest is determined and the amount of the first analyte of interest quantified, the presence (or absence) of the second analyte of interest is determined by performing a 3-dimensional (“3-D”) dose-response surface analysis of the data (also referred to as a “3-D standard ‘curve’”) based on combinations of the first analyte of interest and the second analyte of interest of known concentrations.

The use of dose-response surface analysis to identify significant variables in the optimization of assays, chemical reactions, etc. is the basis of design of experiments (“DOE”). Response surface analysis has also been used to study drug interactions (See, for example in, Civitico, G., Shaw, T., and Locarnini, S., Antimicrob Agents Chemother., 40, 1180-5 (1996)). Such analyses are generally qualitative analyses. In the present invention, such response surfaces are used for quantitative analysis. Any program known in the art can be used in performing the response surface analysis, such as TableCurve-3D (Systat Software, Inc., San Jose, Calif.). Such programs can be used to provide an automated surface-fitting, namely, the equation that best fits the contour of the surface, for quantitative analysis for use in the assays of the present invention.

In a second embodiment, after the test sample containing the at least two analytes of interest is obtained from a subject, the concentration of one of the analytes (which will be referred to as the “first analyte” of interest) is determined. Preferably, in this second embodiment, the first analyte of interest to be detected or quantified is selected from the group consisting of: galactose, glucose, cholesterol, LDL, HDL, choline, lactic acid, uric acid, phosphatidylcholine, acetylcholine, phosphocholine, CDP-choline, lysophosphatidylcholine, triglycerides and sphingomyelin. At least one analyte-specific enzyme, such as, at least one dismutase, dehydrogenase, oxidase, reductase or synthase or a combination of at least one dismutase, dehydrogenase, oxidase, reductase or synthase, is added to the test sample. Examples of at least one analyte-specific enzyme that can be used are selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof. Preferably, the amount of at least one analyte-specific enzyme that can be added to the test sample is from about 0.0001 unit/mL to about 10,000 units/mL.

After the addition of at least one analyte-specific enzyme is added to the test sample, this mixture is then sampled to obtain a first aliquot containing a portion of the first analyte of interest the second analyte of interest and the at least one analyte-specific enzyme from the test sample. At least one first acridinium carboxamide and a first basic solution is then admixed with the first aliquot. The at least one first acridinium carboxamide and the first basic solution added to the first aliquot can be added sequentially. Preferably, the first acridinium carboxamide is an acridinium-9-carboxamide, including optionally an acridinium-9-carboamide having a structure according to formula I shown below:

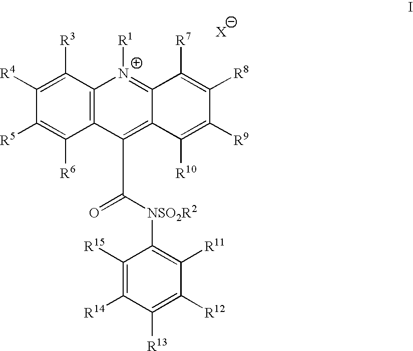

wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl,
   hydroxyl, carboxyl, halogen, halide, nitro, cyano, sulfo, sulfoalkyl, carboxyalkyl and oxoalkyl;
   and further wherein any of the alkyl, alkenyl, alkynyl, aryl or aralkyl may contain one or more heteroatoms; and

optionally, if present, X^{⊖} is an anion.

Methods for preparing acridinium 9-carboxamides are described in Mattingly, P. G. J. Biolumin. Chemilumin., 6, 107-14; (1991); Adamczyk, M.; Chen, Y.-Y., Mattingly, P. G.; Pan, Y. J. Org. Chem., 63, 5636-5639 (1998); Adamczyk, M.; Chen, Y.-Y.; Mattingly, P. G.; Moore, J. A.; Shreder, K. Tetrahedron, 55, 10899-10914 (1999); Adamczyk, M.; Mattingly, P. G.; Moore, J. A.; Pan, Y. Org. Lett., 1, 779-781 (1999); Adamczyk, M.; Chen, Y.-Y.; Fishpaugh, J. R.; Mattingly, P. G.; Pan, Y.; Shreder, K.; Yu, Z. Bioconjugate Chem., 11, 714-724 (2000); Mattingly, P. G.; Adamczyk, M. In Luminescence Biotechnology: Instruments and Applications; Dyke, K. V. Ed.; CRC Press: Boca Raton, pp. 77-105 (2002); Adamczyk, M.; Mattingly, P. G.; Moore, J. A.; Pan, Y. Org. Lett., 5, 3779-3782 (2003); and U.S. Pat. Nos. 5,468,646, 5,543,524, and 5,783,699 (each incorporated herein by reference in their entireties for their teachings regarding same).

The timing and order in which the first acridinium-9-carboxamide is supplied to the first aliquot is not critical provided that it is added after the addition of the at least one analyte-specific enzyme and prior to the addition of at least one first basic solution. The first basic solution will be discussed in more detail below.

After the addition of the first acridinium-9-carboxamide having the structure of formula I to the first aliquot of the test sample, at least one first basic solution is added to the first aliquot of the test sample in order to generate a detectable signal, namely, a first chemiluminescent signal. The first basic solution is a solution that contains at least one base and that has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate and calcium bicarbonate. The amount of basic solution added to the test sample depends on the concentration of the basic solution used in the assay. Based on the concentration of the first basic solution used, one skilled in the art could easily determine the amount of basic solution to be used in the method. Chemiluminescent signals generated can be detected using routine techniques known to those skilled in the art.

Thus, the first chemiluminescent signal generated after the addition of a first basic solution, indicates the presence of the first analyte of interest. The concentration of first analyte of interest is determined from a 3-dimensional (“3-D”) dose-response surface, namely, a 3-D standard “curve”. In this analysis, a combination of the first analyte of interest and the second analyte of interest of known concentrations is used, with the value for the second analyte of interest being 0. Any program known in the art can be used for such response surface analysis, such as TableCurve-3D (Systat Software, Inc., San Jose, Calif.). Such programs can be used to provide an automated surface-fitting, namely, the equation that best fits the contour of the surface, for quantitative analysis for use in the methods of the present invention.

A second sampling of at least one additional aliquot (namely, at least a second aliquot) containing a portion of the first analyte of interest, the second analyte of interest and the at least one analyte-specific enzyme is obtained from the test sample. At least one second acridinium carboxamide and a second basic solution is then admixed with the second aliquot. This second aliquot is then admixed with a second acridinium-9-carboxamide.

The at least one second acridinium carboxamide and the second basic solution added to the second aliquot can be added sequentially. Preferably, the second acridinium carboxamide is an acridinium-9-carboxamide, including optionally an acridinium-9-carboamide having a structure according to formula I shown below:

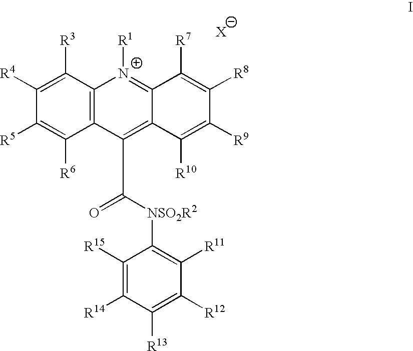

wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl,
   hydroxyl, carboxyl, halogen, halide, nitro, cyano, sulfo, sulfoalkyl, carboxyalkyl and oxoalkyl;
   and further wherein any of the alkyl, alkenyl, alkynyl, aryl or aralkyl may contain one or more heteroatoms; and

optionally, if present, X^{⊖} is an anion.

Methods for preparing acridinium 9-carboxamides are described in Mattingly, P. G. J. Biolumin. Chemilumin., 6, 107-14; (1991); Adamczyk, M.; Chen, Y.-Y., Mattingly, P. G.; Pan, Y. J. Org. Chem., 63, 5636-5639 (1998); Adamczyk, M.; Chen, Y.-Y.; Mattingly, P. G.; Moore, J. A.; Shreder, K. Tetrahedron, 55, 10899-10914 (1999); Adamczyk, M.; Mattingly, P. G.; Moore, J. A.; Pan, Y. Org. Lett., 1, 779-781 (1999); Adamczyk, M.; Chen, Y.-Y.; Fishpaugh, J. R.; Mattingly, P. G.; Pan, Y.; Shreder, K.; Yu, Z. Bioconjugate Chem., 11, 714-724 (2000); Mattingly, P. G.; Adamczyk, M. In Luminescence Biotechnology: Instruments and Applications; Dyke, K. V. Ed.; CRC Press: Boca Raton, pp. 77-105 (2002); Adamczyk, M.; Mattingly, P. G.; Moore, J. A.; Pan, Y. Org. Lett., 5, 3779-3782 (2003); and U.S. Pat. Nos. 5,468,646, 5,543,524, and 5,783,699 (each incorporated herein by reference in their entireties for their teachings regarding same).

The timing and order in which the second acridinium-9-carboxamide is supplied to the second aliquot of the test sample is not critical provided that it is added prior to the addition of at least one second basic solution, which will be discussed in more detail below.

After the addition of the second acridinium-9-carboxamide having the structure according to formula I to the second aliquot of the test sample, at least one second basic solution is added to the second aliquot of the test sample in order to generate a detectable signal, namely, a second chemiluminescent signal. The second basic solution is a solution that contains at least one base and that has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate and calcium bicarbonate. The amount of basic solution added to the second aliquot depends on the concentration of the basic solution used in the assay. Based on the concentration of the first basic solution used, one skilled in the art could easily determine the amount of basic solution to be used in the method. Chemiluminescent signals generated can be detected using routine techniques known to those skilled in the art.

The first acridinium-9-carboxamide and the second acridinium-9-carboxamide used in the assays of the present invention can be the same or different. Likewise, the first basic solution and the second basic solution can be the same or different.

The concentration of the second analyte of interest is determined from the 3-dimensional (“3-D”) dose-response surface or 3-D standard “curve”, using a value for the first analyte of interest that was determined as described above. Preferably, the second analyte of interest is a haloperoxidase. The haloperoxidase can be selected from the group consisting of: myeloperoxidase, thyroperoxidase, eosinoperoxidase, eosinophil peroxidase and lactoperoxidase.

C. Kit for Detecting or Quantifying an Analyte of Interest and for Detecting or Quantifying Haloperoxidase Activity in a Single Test Sample

In another embodiment, the present invention relates to a kit for determining or detecting the presence of at least two analytes of interest in a test sample. In one aspect, the kit can contain at least one acridinium-9-carboxamide having the structure according to Formula I:

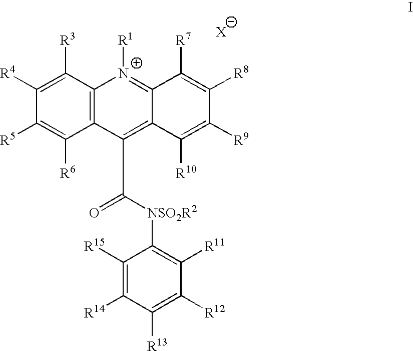

wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl,
   hydroxyl, carboxyl, halogen, halide, nitro, cyano, sulfo, sulfoalkyl, carboxyalkyl and oxoalkyl; and
further wherein any of the alkyl, alkenyl, alkynyl, aryl or aralkyl may contain one or more heteroatoms; and

optionally, if present, X^{⊖} is an anion.

Alternatively, the kit can contain at least two acridinium-9-carboxamides having the structure of the above-described formula I (which can be referred to, for example, as a first acridinium-9-carboxamide, second acridinium-9-carboxamide, etc.). The two or more acridinium-9-carboxamides can be the same or different from each other.

Additionally, the kit can also contain at least one basic solution. Alternatively, the kit can contain at least two basic solutions. The two or more basic solutions can be identical to each other or different from each other.

Furthermore, the kit can also contain at least one analyte-specific enzyme or hydrogen peroxide generating enzyme. The analyte-specific enzyme or hydrogen peroxide generating enzyme that can be used can be selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

Also, the kit can also contain one or more instructions for detecting and quantifying at least two analytes in a test sample. The kit can also contain instructions for generating a standard curve for the purposes of quantifying a first analyte of interest or a reference standard for purposes of quantifying the first analyte of interest in the test sample. Such instructions optionally can be in printed form or on CD, DVD, or other format of recorded media.

Also, the kit can also contain one or more instructions for performing three dimensional dose response surface analysis to calculate the amount of any number of analytes of interest in a test sample. For example, in two analytes of interest are to be quantified in a test sample, the kit can contain one or more instructions for performing the three dimensional dose response analysis for the first analyte of interest, the second analyte of interest or both the first analyte of interest and a second analyte of interest in the test sample. Such instructions optionally can be in printed form or on CD, DVD, or other format of recorded media.

The kit can be used to identify and quantify any number of analytes of interest in a test sample. Preferably, at least one analyte of interest is a haloperoxidase or is an analyte of interest selected from the group consisting of: galactose, glucose, cholesterol, LDL, HDL, choline, lactic acid, uric acid, phosphatidylcholine, acetylcholine, phosphocholine, CDP-choline, lysophosphatidylcholine, triglycerides and sphingomyelin. The haloperoxidase is selected from the group consisting of: myeloperoxidase, thyroperoxidase, eosinoperoxidase, eosinophil peroxidase and lactoperoxidase. More preferably, at least two of the analytes of interest are a haloperoxidase (such as those described above) and analyte of interest selected from the group consisting of: galactose, glucose, cholesterol, LDL, HDL, choline, lactic acid, uric acid, phosphatidylcholine, acetylcholine, phosphocholine, CDP-choline, lysophosphatidylcholine, triglycerides and sphingomyelin.

By way of example, and not of limitation, examples of the present invention shall now be provided.

### EXAMPLE 1

### Creation of a 3-Dimensional Dose-Response Surface for the Analysis of Choline and Myeloperoxidase in a Test Sample

Chemiluminescent Detection Reagent.

9-[[(3-Carboxypropyl) [(4-methylphenyl)sulfonyl]amino]-carbonyl]-10-(3-sulfopropyl)acridinium inner salt was dissolved in reagent grade water containing sodium cholate (0.1% wt/vol) to give a concentration of 250 nM.

Choline Standard Solutions.

Choline standards (0, 5, 10, 20, 30, 50, 75, and 150 mM in phosphate buffer, 0.2 M, pH 8) were prepared as reported in Adamczyk M, Brashear R J, Mattingly P G, Tsatsos P H, Anal Chim Acta., 579(1):61-7 (2006).

Choline Oxidase Reagent.

Choline oxidase (1 U/mL in phosphate buffer, 0.2 M, pH 8; 0.1% sodium cholate, 1 mM methionine, 100 mM sodium chloride).

Standard Solution of Myeloperoxidase.

Myeloperoxidase from human leukocytes (Sigma #M6908) was diluted in phosphate buffered saline (PBS, pH 7.2) containing methionine (1 mM) to give solutions of 2900, 1450, 725, 362.50, 181.25, 90.63, 45.31, 22.66, 11.33, and 0.00 ng/mL.

Protocol.

The standard choline solutions (4 μL) and the standard myeloperoxidase solutions (20 μL) were arrayed in a 96-well microplate as in Table 1 below. The plate was placed in a microplate luminometer (Mithras LB-940, BERTHOLD TECHNOLOGIES U.S.A. LLC, Oak Ridge, Tenn.) at 37° C. Choline oxidase reagent (40 μL) was dispensed into each well and the plate was incubated for 30 minutes. Well by well, the chemiluminescent detection reagent (40 μL) and aqueous sodium hydroxide (0.25 N, 100 μL) were sequentially added and the chemiluminescent signal recorded for 2 seconds.

TABLE 1

| | Choline | | | | | | | | | | | |
|---|---|---|---|---|---|---|---|---|---|---|---|---|
| MPO | 1 | 2 | 3 | 4 | 5 | 6 | 7 | 8 | 9 | 10 | 11 | 12 |
| A | 150/2900 | 150/1450 | 150/725 | 150/362.5 | 150/181.3 | 150/90.7 | 150/45.4 | 150/22.7 | 150/11.4 | 150/5.7 | 150/2.9 | 150/0 |
| B | 75/2900 | 75/1450 | 75/725 | 75/362.5 | 75/181.3 | 75/90.7 | 75/45.4 | 75/22.7 | 75/11.4 | 75/5.7 | 75/2.9 | 75/0 |
| C | 50/2900 | 50/1450 | 50/725 | 50/362.5 | 50/181.3 | 50/90.7 | 50/45.4 | 50/22.7 | 50/11.4 | 50/5.7 | 50/2.9 | 50/0 |
| D | 30/2900 | 30/1450 | 30/725 | 30/362.5 | 30/181.3 | 30/90.7 | 30/45.4 | 30/22.7 | 30/11.4 | 30/5.7 | 30/2.9 | 30/0 |
| E | 20/2900 | 20/1450 | 20/725 | 20/362.5 | 20/181.3 | 20/90.7 | 20/45.4 | 20/22.7 | 20/11.4 | 20/5.7 | 20/2.9 | 20/0 |
| F | 10/2900 | 10/1450 | 10/725 | 10/362.5 | 10/181.3 | 10/90.7 | 10/45.4 | 10/22.7 | 10/11.4 | 10/5.7 | 10/2.9 | 10/0 |
| G | 5/2900 | 5/1450 | 5/725 | 5/362.5 | 5/181.3 | 5/90.7 | 5/45.4 | 5/22.7 | 5/11.4 | 5/5.7 | 5/2.9 | 5/0 |
| H | 0/2900 | 0/1450 | 0/725 | 0/362.5 | 0/181.3 | 0/90.7 | 0/45.4 | 0/22.7 | 0/11.4 | 0/5.7 | 0/2.9 | 0/0 |

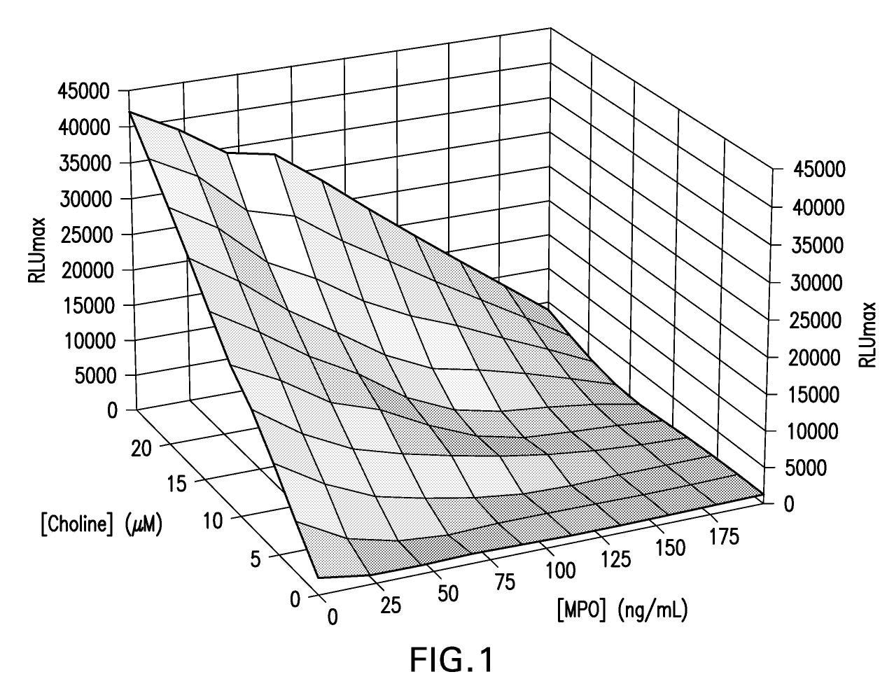

The resulting signal at each choline/myeloperoxidase concentration is tabulated in Table 2 below, and the resulting 3-dimensional (3D) dose-response surface is graphically shown in FIG. 1.

TABLE 2

| Choline | MPO (ng/mL) | | | | | | | | | | | |
|---|---|---|---|---|---|---|---|---|---|---|---|---|
| (μmol/mL) | 2900.0 | 1450.0 | 725.0 | 362.5 | 181.3 | 90.6 | 45.3 | 22.7 | 11.3 | 5.7 | 2.8 | 0.0 |
| 25.0 | 640 | 1270 | 1230 | 1170 | 7850 | 27870 | 34340 | 38260 | 40350 | 38940 | 44300 | 42070 |
| 12.5 | 1160 | 1190 | 1160 | 1180 | 1190 | 6410 | 13730 | 18030 | 20020 | 20370 | 23470 | 21700 |
| 8.3 | 1480 | 1220 | 1240 | 1150 | 1180 | 4740 | 8240 | 11550 | 13800 | 14730 | 15530 | 16190 |
| 5.0 | 1450 | 1300 | 1200 | 1150 | 1110 | 1470 | 3830 | 6700 | 8420 | 9670 | 11230 | 10620 |
| 3.3 | 1390 | 1280 | 1270 | 1220 | 1170 | 1260 | 1830 | 3920 | 5490 | 6510 | 7290 | 7800 |
| 1.7 | 1290 | 1290 | 1200 | 1220 | 1110 | 1200 | 1290 | 2010 | 3440 | 3940 | 4630 | 4950 |
| 0.8 | 1260 | 1320 | 1210 | 1210 | 1120 | 1230 | 1250 | 1510 | 2360 | 3020 | 3430 | 3600 |
| 0.0 | 1320 | 1300 | 1200 | 1250 | 1240 | 1220 | 1220 | 1240 | 1440 | 1770 | 1990 | 2270 |

### EXAMPLE 2

### Interdependent Assay for Choline and Myeloperoxidase in a Test Sample

A standard sample containing both choline and myeloperoxidase was first analyzed for the concentration of choline present according to the protocol reported in Adamczyk M, Brashear R J, Mattingly P G, Tsatsos P H., Anal Chim Acta., 579(1):61-7 (2006).

The sample was then analyzed for the concentration of myeloperoxidase present according to the following protocol:

The test sample (24 μL) was dispensed into the well of a microplate which was then placed in a microplate luminometer (Mithras LB-940, BERTHOLD TECHNOLOGIES U.S.A. LLC, Oak Ridge, Tenn.) at 37° C. Choline oxidase reagent (40 μL) was dispensed into the well and the plate was incubated for 30 minutes. The chemiluminescent detection reagent (40 μL) and aqueous sodium hydroxide (0.25 N, 100 μL) were sequentially added and the chemiluminescent signal was recorded for 2 seconds.

The concentration of myeloperoxidase present was determined from a polynomial equation describing the surface dose-response generated in Example 1. The results are shown below in Table 3.

TABLE 3

| | | | [MPO] | |
|---|---|---|---|---|
| [Choline] | | [MPO] (ng/mL) | (ng/mL) | |
| (μM) | MPO_RLUmax | Calculated | Actual | % Difference |
| 8.3 | 1,180 | 177.2 | 181.3 | −2.2 |
| 8.3 | 4,740 | 87.9 | 90.6 | −3.0 |
| 8.3 | 8,240 | 46.03 | 45.3 | 1.6 |
| 8.3 | 11,550 | 22.9 | 22.7 | 1.1 |
| 8.3 | 13,800 | 12.6 | 11.3 | 11.2 |
| 8.3 | 15,530 | 3 | 2.8 | 7.3 |
| 12.5 | 1,190 | 168.6 | 181.3 | −7.0 |
| 12.5 | 6,410 | 93 | 90.6 | 2.6 |
| 12.5 | 13,730 | 43.5 | 45.3 | −4.0 |
| 12.5 | 18,030 | 21 | 22.7 | −9.5 |
| 12.5 | 20,020 | 13.1 | 11.3 | 15.6 |

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. An interdependent method for detecting at least two analytes of interest in a test sample, the method comprising the steps of:
a) contacting a test sample containing a first analyte of interest and a second analyte of interest with at least one analyte-specific enzyme, wherein the first analyte of interest is selected from the group consisting of: galactose, glucose, cholesterol, LDL, HDL, choline, lactic acid, uric acid, phosphatidylcholine, acetylcholine, phosphocholine, CDP-choline, lysophosphatidylcholine, triglycerides and sphingomyelin;
b) adding an acridinium-9-carboxamide to the test sample;
c) adding a basic solution to the test sample to generate a light signal;
d) measuring the light generated from the light signal and calculating the amount of first analyte of interest present in the test sample; and
e) performing a three dimensional dose response surface analysis to calculate the amount of the second analyte of interest in the test sample.

2. The method of claim 1, wherein the analyte-specific enzyme is a dismutase, dehydrogenase, oxidase, reductase or synthase or a combination of at least one dismutase, dehydrogenase, oxidase, reductase or synthase.

3. The method of claim 2, wherein the analyte-specific enzyme is selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3- phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6- oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N- acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl- alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

4. The method of claim 1, wherein the second analyte of interest is a haloperoxidase.

5. The method of claim 4, wherein the haloperoxidase is selected from the group consisting of: myeloperoxidase, thyroperoxidase, eosinoperoxidase, eosinophil peroxidase and lactoperoxidase.

6. The method of claim 1, wherein the test sample is whole blood, serum or plasma.

7. The method of claim 1, further comprising quantifying the amount of the first analyte of interest in the test sample by relating the amount of light generated in the test sample by comparison to a standard curve for said analyte.

8. The method of claim 6, wherein the standard curve is generated from solutions of an analyte of a known concentration.

9. The method of claim 1, further comprising quantifying the activity of amount of the second analyte of interest by using a combination of known concentrations of the first analyte of interest and the second analyte of interest.

10. The method of claim 1, wherein the acridinium-9 - carboxamide has a structure according to formula I: 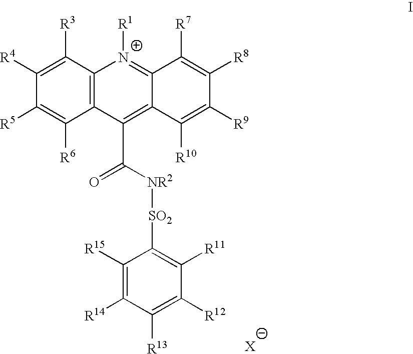
wherein R¹ and R² are each independently selected from the group consisting of: alkyl, alkenyl, alkynyl, aryl or aralkyl, sulfoalkyl and carboxyalkyl, and
wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, and carboxyalkyl; and
optionally, if present, X⁶³ is an anion.

11. An interdependent method for detecting at least at least two analytes of interest in a test sample, the method comprising the steps of:
a) adding an acridinium-9-carboxamide to a test sample;
b) generating in or providing to the test sample a source of hydrogen peroxide before or after the addition of an acridinium-9-carboxamide;
c) adding a basic solution to the test sample to generate a light signal;
d) measuring the light generated from the light signal and calculating the amount of a first analyte of interest present in the test sample, wherein the first analyte of interest is a haloperoxidase; and
e) performing a three dimensional dose response surface analysis to calculate the amount of the second analyte of interest in the test sample.

12. The method of claim 11, wherein the second analyte of interest is selected from the group consisting of: galactose, glucose, cholesterol, LDL, HDL, choline, lactic acid, uric acid, phosphatidylcholine, acetylcholine, phosphocholine, CDP-choline, lysophosphatidylcholine, triglycerides and sphingomyelin.

13. The method of claim 11, wherein the haloperoxidase is selected from the group consisting of: myeloperoxidase, thyroperoxidase, eosinoperoxidase, eosinophil peroxidase and lactoperoxidase.

14. The method of claim 11, wherein the test sample is whole blood, serum or plasma.

15. The method of claim 11, further comprising quantifying the amount of the first analyte of interest in the test sample by relating the amount of light generated in the test sample by comparison to a standard curve for said analyte.

16. The method of claim 15, wherein the standard curve is generated from solutions of an analyte of a known concentration.

17. The method of claim 11, further comprising quantifying the activity of amount of the second analyte of interest by using a combination of known concentrations of the first analyte of interest and the second analyte of interest.

18. The method of claim 11, wherein the acridinium-9 - carboxamide has a structure according to formula I: 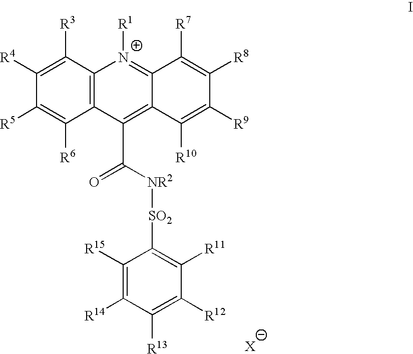
wherein R¹ and R² are each independently selected from the group consisting of: alkyl, alkenyl, alkynyl, aryl or aralkyl, sulfoalkyl and carboxyalkyl, and
wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, and carboxyalkyl; and
optionally, if present, X^{⊖}is an anion.

19. The method of claim 11, wherein the hydrogen peroxide is provided by adding a buffer or a solution containing hydrogen peroxide.

20. The method of claim 11, wherein the hydrogen peroxide is generated by adding a hydrogen peroxide generating enzyme to the test sample.

21. An interdependent method for detecting at least at least two analytes of interest in a test sample, the method comprising the steps of:
a) contacting a test sample containing a first analyte of interest and a second analyte of interest with at least one analyte-specific enzyme;
b) sampling the test mixture to obtain a first aliquot containing a portion of the first analyte of interest and the second analyte of interest from the test sample;
c) adding a first acridinium-9-carboxamide to the first aliquot;
d) adding a first basic solution to the first aliquot to generate a light signal;
e) measuring the light generated from the light signal;
f) sampling the test mixture to obtain a second aliquot containing a portion of the first analyte of interest and the second analyte of interest from the test sample;
g) adding a second acridinium-9-carboxamide to the second aliquot;
h) adding a second basic solution to the second aliquot to generate a light signal;
i) measuring the light generated from the light signal in step h); and
j) performing a three dimensional dose response surface analysis using the using the amount of light measured in steps e) and i)) to calculate the amount of the first and second analytes of interest in the test sample.

22. The method of claim 21, wherein the first analyte of interest is selected from the group consisting of: galactose, glucose, cholesterol, LDL, HDL, choline, lactic acid, uric acid, phosphatidylcholine, acetylcholine, phosphocholine, CDP-choline, lysophosphatidylcholine, triglycerides and sphingomyelin.

23. The method of claim 21, wherein the analyte-specific enzyme is a dismutase, dehydrogenase, oxidase, reductase or synthase or a combination of at least one dismutase, dehydrogenase, oxidase, reductase or synthase.

24. The method of claim 23, wherein the analyte-specific enzyme is selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3- phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6- oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N- acyihexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl- alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

25. The method of claim 21, wherein the second analyte of interest is a haloperoxidase.

26. The method of claim 25, wherein the haloperoxidase is selected from the group consisting of: myeloperoxidase, thyroperoxidase, eosinoperoxidase, eosinophil peroxidase and lactoperoxidase.

27. The method of claim 21, wherein the test sample is whole blood, serum or plasma.

28. The method of claim 21, wherein the first acridinium-9- carboxamide and second acridinium-9-carboxamide each have a structure according to formula I: 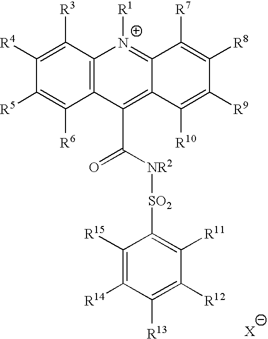
wherein R¹ and R² are each independently selected from the group consisting of: alkyl, alkenyl, alkynyl, aryl or aralkyl, sulfoalkyl and carboxyalkyl, and
wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, and carboxyalkyl; and
optionally, if present, X⁶³ is an anion.

29. A kit for use in detecting at least two analytes of interest in a test sample, the kit comprising:
a. at least one acridinium-9-carboxamide;
b. at least one basic solution;
c. at least one analyte-specific enzyme or hydrogen peroxide generating enzyme;
d. instructions for detecting the amount of at least one analyte of interest in a test sample; and
e. instructions for performing a dimensional dose response surface analysis to calculate the amount of at least one analyte of interest in the test sample.

30. The kit of claim 29, wherein the at least one analyte-specific enzyme or hydrogen peroxide generating enzyme is a dismutase, dehydrogenase, oxidase, reductase or synthase or a combination of at least one dismutase, dehydrogenase, oxidase, reductase or synthase.

31. The kit of claim 30, wherein the at least one analyte-specific enzyme or hydrogen peroxide generating enzyme is selected from the group consisting of: (R)- 6-hydroxynicotine oxidase, (S)-2-hydroxy acid oxidase, (S)-6-hydroxynicotine oxidase, 3-aci- nitropropanoate oxidase, 3-hydroxyanthranilate oxidase, 4-hydroxymandelate oxidase, 6- hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono- 1,4-lactone oxidase, D-arabinono- 1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethyiglycine oxidase, D-manrntol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L- glutamate oxidase, L-gulonolactone oxidase, L-lysine 6-oxidase, L-lysine oxidase, long-chain- alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan cxj3-oxidase, urate oxidase (uricase, uric acid oxidase), vanillyl-alcohol oxidase, xanthine oxidase, xylitol oxidase and combinations thereof.

32. The kit of claim 29, wherein the acridinium-9-carboxamide has a structure according to formula I: 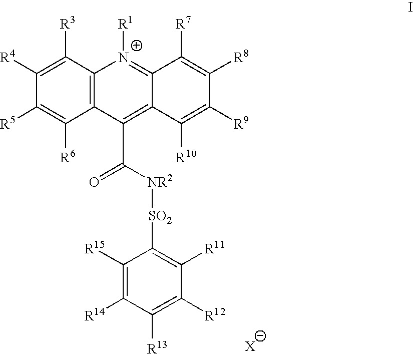
wherein R¹ and R² are each independently selected from the group consisting of: alkyl, alkenyl, alkynyl, aryl or aralkyl, sulfoalkyl and carboxyalkyl, and
wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, and carboxyalkyl; and
optionally, if present, X^{⊖}is an anion.

33. The kit of claim 29, wherein at least one analyte is selected from the group consisting of: galactose, glucose, cholesterol, LDL, HDL, choline, lactic acid, uric acid, phosphatidylcholine, acetylcholine, phosphocholine, CDP-choline, lysophosphatidylcholine, triglycerides and sphingomyelin.

34. The kit of claim 29, wherein at least one analyte is a haloperoxidase.

35. The kit of claim 34, wherein the haloperoxidase is selected from the group consisting of: myeloperoxidase, thyroperoxidase, eosinoperoxidase, eosinophil peroxidase and lactoperoxidase.

36. A kit for use in detecting at least two analytes of interest in a test sample, the kit comprising:
a. at least two acridinium-9-carboxamides;
b. at least two basic solutions;
c. at least one analyte-specific enzyme or hydrogen generating enzyme; and
d. instructions for performing a dimensional dose response surface analysis to calculate the amount of at least two analytes of interest in the test sample

37. The kit of claim 36, wherein the at least one analyte-specific enzyme or hydrogen generating enzyme is a dismutase, dehydrogenase, oxidase, reductase or synthase or a combination of at least one dismutase, dehydrogenase, oxidase, reductase or synthase.

38. The kit of claim 37, wherein the at least one analyte-specific enzyme or hydrogen peroxide generating enzyme is selected from the group consisting of: (R)- 6-hydroxynicotine oxidase, (S)-2-hydroxy acid oxidase, (S)-6-hydroxynicotine oxidase, 3-aci- nitropropanoate oxidase, 3-hydroxyanthranilate oxidase, 4-hydroxymandelate oxidase, 6- hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono- 1,4-lactone oxidase, D-arabinono- 1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L- glutamate oxidase, L-gulonolactone oxidase, L-lysine 6-oxidase, L-lysine oxidase, long-chain- alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

39. The kit of claim 36, wherein each of the acridinium-9- carboxamides has a structure according to formula I: 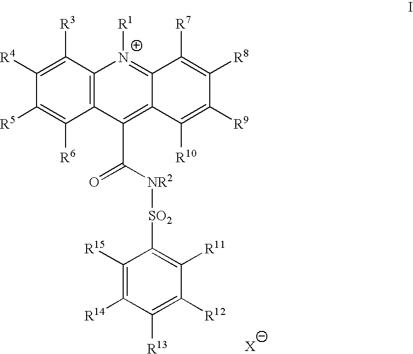
wherein R¹ and R² are each independently selected from the group consisting of: alkyl, alkenyl, alkynyl, aryl or aralkyl, sulfoalkyl and carboxyalkyl, and
wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, and carboxyalkyl; and
optionally, if present, X^{⊖}is an anion.

40. The kit of claim 36, wherein at least one analyte is selected from the group consisting of: galactose, glucose, cholesterol, LDL, HDL, choline, lactic acid, uric acid, phosphatidylcholine, acetylcholine, phosphocholine, CDP-choline, lysophosphatidylcholine, triglyceride and sphingomyelin.

41. The kit of claim 36, wherein at least one analyte is a haloperoxidase.

42. The kit of claim 41, wherein the haloperoxidase is selected from the group consisting of: myeloperoxidase, thyroperoxidase, eosinoperoxidase, eosinophil peroxidase and lactoperoxidase.

43. The kit of claim 36, wherein at least two acridinium-9- carboxamides are each different from one another.

44. The kit of claim 36, wherein the at least two acridinium-9- carboxamides are the same.

45. The kit of claim 36, wherein the at least two basic solutions are different from each other.

46. The kit of claim 36, wherein the at least two basic solutions are the same.
